(19) United States
(12) Patent Application Publication
(10) Pub. No.: US 2005/0191338 A1
(43) Pub. Date: Sep. 1, 2005

(54) Transdermal drug delivery composition comprising a small molecule gel and process for the preparation thereof

(76) Inventors: Lifeng Kang, Singapore (SG); Prashant D. Sawant, Singapore (SG); Xiang Yang Liu, Singapore (SG); Sui Yung Chan, Singapore (SG)

Correspondence Address: KLARQUIST SPARKMAN, LLP, 121 SW SALMON STREET, SUITE 1600, PORTLAND, OR, 97204, US

(21) Appl. No.: 11/046,419

(22) Filed: Jan. 28, 2005

Related U.S. Application Data
(60) Provisional application No. 60/540,872, filed on Jan. 30, 2004.

(51) Int. Cl. A61K 31/56; A61K 31/445; A61K 9/70

(52) U.S. Cl. 424/449; 514/182; 514/317

(57) ABSTRACT

This invention relates to a transdermal drug delivery composition comprising a small molecule gel, which small molecule gel comprises a small molecule gelling agent and a solvent, in which solvent is dissolved or suspended (a) a drug that is suitable for transdermal delivery and (b) a skin permeation enhancer. The invention also relates to a process for the preparation of the transdermal drug delivery composition described above.

### CROSS-REFERENCE TO RELATED APPLICATIONS

This application claims benefit of U.S. Provisional Patent application Ser. No. 60/540,872, filed. Jan. 30, 2004, entitled “TRANSDERMAL DRUG DELIVERY COMPOSITION COMPRISING AN ORGANOGEL AND PROCESS FOR THE PREPARATION THEREOF”, the contents of which are hereby incorporated by reference.

### FIELD OF THE INVENTION

The present invention relates to transdermal drug delivery compositions comprising small molecule gels, and to processes for their preparation.

### BACKGROUND OF THE INVENTION

The transdermal delivery of drugs has been discussed in a number of journal references, for example:
Brown L., Langer R., (1988), Transdermal delivery of drugs, Annu. Rev. Med., 39, 221-229
Groves J. E., Mandel M. R., (1975), The long-acting phenothiazines, Arch. Gen. Psychiatry, 32, 893-900.
Samanta M. K., Dube R., Suresh B., (2003), Transdermal drug delivery system of haloperidol to overcome self-induced extrapyramidal syndrome, Drug Dev. Ind. Pharm., 29, 405-415
Silver A. A., Shytle R. D., Philipp M. K., Wilkinson B. J., McConville B., Sanberg P. R., (2001), Transdermal nicotine and haloperidol in Tourette's disorder: a double-blind placebo-controlled study, J. Clin. Psychiatry, 62, 707-714
Tuninger E., Levander S., (1996), Large variations of plasma levels during maintenance treatment with depot neuroleptics, Br. J. Psychiatry, 169, 618-621
Vaddi H. K., Ho P. C., Chan S. Y., (2002), Terpenes in propylene glycol as skin-penetration enhancers: permeation and partition of haloperidol, Fourier transform infrared spectroscopy, and differential scanning calorimetry, J. Pharm. Sci., 91, 1639-1651
Vaddi H. K., Ho P. C., Chan Y. W., Chan S. Y., (2003), Oxide terpenes as human skin penetration enhancers of haloperidol from ethanol and propylene glycol and their modes of action on stratum corneum, Biol. Pharm. Bull., 26, 220-228
Whitehead T., (1975), Letter: long-acting phenothiazines, Br. Med. J., 2, 502,

The transdermal delivery of drugs has also been disclosed in the following patents:
U.S. Pat. No. 6,572,879 to Yum et al.
U.S. Pat. No. 6,586,000 to Luo et al.
U.S. Pat. No. 6,602,912 to Hsu et al.
U.S. Pat. No. 6,620,435 to Osborne et al.

In all of the above references, the transdermal drug delivery compositions are composed of drug solutions that are held within a vehicle. In one of the above references (Brown et al.), covalently crosslinked polymers having large molecular weights are used as the vehicle. In some instances, polymeric gels have been found to have disadvantages, such as staining the skin on which they are applied.

Recently, it has been found that the aggregation of low molecular weight compounds in organic solvents can result in the formation of small molecule gels. These small molecule gels consist of self-organised three-dimensional interconnecting networks, which networks immobilise liquids.

### SUMMARY OF THE INVENTION

The small molecule gels described in the present invention lend themselves well as replacements to the large molecular weight crosslinked-polymers previously used to prepare transdermal drug delivery compositions.

In one aspect, the present invention provides a transdermal drug delivery composition comprising a small molecule gel, which small molecule gel comprises a small molecule gelling agent and a solvent, in which solvent is dissolved or suspended (a) a drug that is suitable for transdermal delivery and (b) a skin permeation enhancer.

In another aspect, the present invention provides a process for preparing the transdermal drug delivery composition described above, comprising (a) heating a mixture of a small molecule gelling agent and a solvent to dissolve the gelling agent in the solvent; (b) cooling the mixture to form a gel; and (c) adding a drug that is suitable for transdermal delivery and a skin permeation enhancer to the mixture either before, during or after the heating, but prior to cooling.

The transdermal drug delivery compositions described above are advantageous as they can comprise organic solvents and gelling agents that have been found to be safe for application on human skin in the field of cosmetics and food. In addition, gels comprising small molecule gels are less prone than crosslinked polymer gels to stain skin tissues.

### DESCRIPTION OF THE FIGURES

Specific embodiments of the invention are described further through reference to the following figures:

FIG. 1 graphically displays the results of a Dynamic Time Sweep experiment for a small molecule gel comprising 5% w/v GP-1 in propylene glycol.

FIG. 2 graphically displays a Dynamic strain analysis of a small molecule gel comprising 5% w/v GP-1 in propylene glycol.

FIG. 3 displays a standard set up of a flow-through cell for measuring in vitro skin permeation values.

### DETAILED DESCRIPTION OF THE PREFERRED EMBODIMENTS

Small Molecule Gels

Small molecule gels generally comprise arrays of small molecule gelling agents (SMGAs) that are interconnected to each other via fibril branching. The SMGA molecules self-assemble through specific interactions, allowing preferential one-dimensional growth of fibrils. The elongated fibrils can then join to form three-dimensional networks that form the final gel. As fibril branching of the small molecule gelling agents occurs in a liquid medium (solvent), the three-dimensional networks encapsulate the solvent and inhibit its flow. The precise molecular arrangement of the molecules, and consequently the macroscopic qualities of the networks, are determined by the characteristics of the small molecule gelling agent and the nature of the solvent.

The gels prepared from SMGAs differ from the gels comprising crosslinked polymers in that they are held together by non-covalent forces, such as hydrogen bonding, Van der Waals forces, ππ interactions and ionic bonding. Since all these forces are reversible in nature, the gels prepared from SMGAs are usually themselves thermo-reversible. In this invention, the thermo-mechanical control over the micro/nanostructure of the small molecule gel is an important aspect of the regulation of drug permeation. Thermo-reversibility can be defined as the ability of a gel to change between a liquid state and a gel state when it is heated above or cooled below the gel transition temperature.

When a small molecule gel is heated, there is weakening in the interaction between the SMGA molecules that form the gel, even when the temperature reached is below the gel transition temperature. This weakening of the interactions leads to a composition where the liquid component of the small molecule gel is not as strongly held within the gel. When a transdermal drug delivery composition comprises a small molecule gel instead of a crosslinked polymer, increases in temperature such as caused by contacting human skin will lead to a weakening of the gel and a consequent partial liberation of solvent. As the drug is comprised within the solvent, this liberation of the solvent increases the drug contact with the skin, leading to higher drug permeation levels.

In addition to having a micro/nanostructure that is responsive to changes in temperature, the small molecule gels are also advantageous over traditional systems in that (i) they are made up of molecules that have lower molecular weights, which leads to a faster degradation (comparatively to crosslinked polymers), and (ii) they are easy to prepare (comparatively to polymeric and surfactant gels).

Viscoelasticity can be used to assess the quality of the small molecule gels used in the present invention, and this characteristic can be quantitatively measured as the “limit of linearity, γₒ”. The value for γₒ is calculated by applying different forces to the small molecule gel, and by measuring the elastic modulus (G*) and the storage modulus (G′) values obtained. The relationship between the G* and G′ is given by the equation G*=G′+iG″ where G′ describes the elastic qualities of the substance, G″ (loss modulus) describes the viscosity qualities of the substance and i is a complex number \sqrt{−1}=i. The elastic modulus is a measure of overall resistance to deformation. The strain applied affects the 3D interconnecting microstructure of the gel, and at a specific level of strain, which is defined as γₒ, the network structures begins to break down, which is identified by a decrease of the elastic modulus. Until an amount of strain equal to γₒ is attained, the elastic modulus of the gel is unchanged and it usually provides a linear response that is parallel to the X-axis, as the strain is increased. A higher γₒ value indicates that the microstructures in the small molecule gel are more resistant to deformation when strain is applied, which is usually due to the higher number of discrete fibres in the small molecule gel. A high value for γₒ is usually indicative of a stronger small molecule gel.

In some embodiments, the porosity of the small molecule gels is 500 nm or less, while in other embodiments the porosity is in the range of from about 50 nm to about 500 nm, from about 200 nm to about 500 nm, or from about 50 nm to about 300 nm. It is not essential, however, that the gels have porosity within these ranges.

Small Molecule Gelling Agents

Small Molecule Gelling Agents differ from the polymeric materials traditionally used to form gels in that they have low molecular weights, usually of 3000 g/mol or less, and in some cases molecular weights of 1000 g/mol or less. SMGAs are known to have varied structural and chemical elements, and examples of suitable SMGAs include, but are not limited to the following classes of molecules: fatty acid derivatives, steroid derivatives such as D-3β-hydroxy-17,17-dipropyl-17a-azahomoandrostanyl-17a-oxy (STNO) and D-3β-hydroxy-17,17-dipropyl-17a-azahomoandrostanyl-17a-aza (STHN), gelling agents containing steroidal and condensed aromatic rings, such as anthryl and anthraquinone appended steroid-based gelling agents, for example 2,3-bis-n-decyloxyanthracene (DDOA) and 2,3-bis-n-decyloxyanthraquinone (DDOA), azobenzene steroid-based gelling agents, such as molecules having a highly polar azobenzene group linked at C3 of a steroidal moiety, amino acid-type SMGAs, and organometallic compounds, such as mononuclear copper β-diketonates. Organometallic compounds must be selected with care, to insure that they do not cause an increased degradation of the transdermal drug held within the small molecule gel. In some embodiments, two or more gelling agents can be used in combination to prepare the small molecule gels. Specific examples of the above classes of compounds and other known gelling agents can be found, for example, in Low Molecular Mass Gelators of Organic Liquids and the Properties of Their Gels, by Pierre Terech and Richard G. Weiss (Chem. Rev. 1997, 97, 3133-3159), and in Organogels and Low Molecular Mass Organic Gelators, by David J. Abdallah and Richard G. Weiss (Adv. Matter. 2000, 12, No. 17, 1237-1247), the contents of which references are hereby incorporated by reference.

As the small molecule gels used in the present invention are intended to come into contact with skin, it is preferable that the SMGA used to form the small molecule gel be biocompatible. By biocompatible is meant a SMGA that is, for example, non-toxic. Preferably, the SMGA also will not elicit any unwanted reactions with the skin, such as burns or rashes. Examples of biocompatible SMGAs include many amide based SMGA molecules described in the Pierre Terech and Richard G. Weiss reference (see above). Examples of preferred biocompatible SMGAs include N-laurolylglutamic acid dibutyl amide (GP-1) and 1,3:2,4-bis-O-benzylidene-D-sorbitol (D-DBS). These SMGAs have been shown to be suitable for preparing small molecule gels in references such as Sawant et al., Chem. Mater., 2002, 14, 3793-3798 (GP-1); Yamasaki et al., Bull, Chem. Soc. Jpn., 1995, 68, 123-127; Yamasaki et al., Bull, Chem. Soc. Jpn., 1995, 68, 146-151; Yamasaki et al., Bull, Chem. Soc. Jpn., 1994, 67, 906-911; and Yamasaki et al., Bull, Chem. Soc. Jpn., 1994, 67, 2053-2056; (D-DBS), which references are incorporated herewith by reference.

The concentration of small molecule gelling agent in the mixture is preferably from about 0.1% w/v to about 20% w/v, more preferably from about 4% w/v to about 15% w/v. It is not essential, however, that the concentrations of small molecule gelling agent fall within these ranges.

Solvents

Small molecule gels have been prepared with a large variety of solvents. Examples of such suitable solvents include, without limitation, propylene glycol, ethyl acetate, ethanol, 1-propanol, 1-butanol, 1-octanol, benzyl alcohol, triethylsilane, trimethylchlorosilane, diemthylpolysiloxane, glycerol and water. As with the gelling agents, two or more solvents can be combined to prepare a small molecule gel. Additional examples of solvents are found in the reference by Terech and Weiss (1997). The solvent is preferably not an irritant or an allergen, and it is also preferably non-toxic. As the small molecule gels used to prepare the transdermal drug delivery composition of the invention will come into contact with skin, it is preferable that the solvent used to prepare the small molecule gel also be biocompatible. When selecting a solvent, other considerations, such as the solvent's effect on the transdermal rate of drug delivery, is preferably taken into consideration.

Preferably, the solvent used to prepare the small molecule gel is propylene glycol or ethanol. Propylene glycol and ethanol have been used in the past for the preparation of skin penetration enhancer solutions, and they are common solvents or co-solvents for drugs or excipients in pharmaceutical formulations. Propylene glycol is commonly used as a solvent for terpenoid substances, which are used as skin penetration enhancers in transdermal research and it has well-established levels of systemic toxicity and skin tolerability. Propylene glycol and ethanol were also reported to have synergistic effect with some terpenes for the permeation enhancement of drugs.

Drugs Suitable for Transdermal Delivery

Drugs that are suitable for transdermal delivery are known in the art, and any such drug can be used in the transdermal drug delivery composition of the present invention. Examples of suitable drugs include, for example, haloperidol, norgestimate, pergolide, racemic phenylpropanolamine, (+)-norephedrine, (−)-norephedrine, (+)-norpseudoephedrine, (−)-norpseudoephedrine, acyclovir and ethyinyl estradiol, of which haloperidol is preferred.

Haloperidol (HP) and other similar drugs are suitable candidates for the development of transdermal dosage forms. There is a clinical need for HP to be so formulated to maintain therapeutic plasma levels. Haloperidol decanoate (HPD) is currently the only commercially available long-acting formulation of Haloperidol. However, HPD treatments take the form of oily injections that can cause pain at the site of injection due to slow dispersion, and the treatments require a complex dosing scheme to accommodate the different pharmacokinetic profiles of HP and HPD. Individual differences in esterases also cause problems by producing non-uniform release of free HP. This depot formulation has been shown to display marked plasma concentration variations, which are clinically undesirable. The above disadvantages with traditional HP treatments can be avoided through the transdermal drug delivery compositions of the present invention, which offer alternatives in long-acting formulations.

In some embodiments, the drug comprised in the transdermal drug delivery system of the invention is dissolved in the solvent that forms the small molecule gel. Preferably, the solvent is saturated with the drug so that the permeation of the drug through the skin is maximised.

Preferably, the concentration of the drug in the small molecule gel is from 3 mg/ml to 6 mg/ml. The optimal level of drug in the small molecule gel is dependent on the nature of the drug, the solvent and of the SMGA, and also on the presence of a skin permeation enhancer.

Transdermal Delivery Enhancers

For many drugs, the permeation rate through skin is low, and as such a large contact area on the skin is required for suitable administration levels. In order to circumvent this low permeation rate, transdermal delivery enhancers (also referred to as skin permeation enhancers, or simply as enhancers) can be incorporated into transdermal delivery compositions. A variety of enhancers have been used in the past to achieve higher permeation rates for polymeric gels, and any of these enhancers can be used in the present invention. Examples of enhancers that are suitable for use in the present invention include terpene-based molecules. Terpenes have been shown to be excellent skin penetration enhancers for both lipophilic and hydrophilic drugs. They have a low toxicity and a low skin irritancy, and they have been recognized as safe (GRAS) by the FDA (Godwin & Michniak 1999; Williams & Barry 1991). Terpenes are obtained from plant essential oils and they comprise isoprene units (C₅H₈). Terpenes are widely used in perfumes, flavourings and medicines.

Specific examples of suitable terpenoid enhancers include linalool, menthol, cineole and farnesol. Preferably, the concentration of enhancer in the small molecule gel is from 1 to 10% (w/v).

In some embodiments, it has been found that certain enhancers can also act as branching agents during preparation of the gel. When using certain enhancers with specific combinations of SMGAs and solvents, such as linalool (enhancer) with GP-1 (SMGA) and propylene glycol (solvent), the quality of the resulting gel is enhanced in a manner similar to that seen when using branching agents. Branching agents and their effects are described below.

Branching Agents

The small molecule gels that are found in the transdermal drug delivery composition of the invention can also optionally comprise a branching agent.

Recently, it was discovered that small molecule candidates that proved to be unsuitable for forming gels through traditional gelling processes could be transformed into small molecule gels through the use of branching agents (WO 072675 to Liu et al., the contents of which are hereby incorporated by reference). Branching agents have also been used to obtain small molecule gels from specific combinations of SMGAs and solvents that failed to provide small molecule gels under standard gelling procedures. When treated with traditional processes, the unsuccessful SMGAs crystallises out of solution instead of forming clear gels, resulting in opaque gels or pastes that have poor rheological properties and transparency. These poor properties are due to the fact that needle-like crystallites are formed instead of the three-dimensional networks required to form the gel. In some systems, the introduction of a branching agent (also referred to as branching additive) to the mixture comprising the SMGA and the solvent leads to three-dimensional interconnecting network structures instead of crystalline needles. In addition to permitting the gelation of poor gelling agents or the gelation of poor SMGA/solvent combinations, the addition of branching agents has a beneficial effect on the quality of small molecule gels prepared with components known to form gels under standard conditions.

Examples of suitable branching additives include, for example, ethylvinyl/ethylvinyl acetate copolymer (EVACP)[approximate molecular weight of 100,000], which is available from Sp² Scientific Polymer Products Inc, and poly(methyl vinyl ether)/maleic anhydride copolymer [approximate molecular weight of 1,080,000], which is commercially available as Gantrez AN-139, from ISP Europe.

Additional examples of branching agents are also found in the reference by Liu et al. The reference by Liu et al. also provides the guidelines necessary for the selection and the preparation of suitable branching agents. In addition to being non-toxic, the branching agent is preferably selected so that it does not increase the decomposition rate of the transdermal drug.

The concentration of branching agent in the small molecule gel mixture is preferably above 0.001% w/v, and preferably from about 0.001% w/v to about 0.1% w/v. It is not essential, however, that the concentration of the branching agent be within these ranges. The upper limit given in the above preferred range may be exceeded, as in some embodiments increases in the concentration of the branching agent simply leads to thinner fibres and to smaller pore sizes in the gel. The concentration of the branching agent can thus be controlled to obtain gels having required porosity or mechanical properties.

Preparation of the Transdermal Drug Delivery Compositions

The present invention also provides a process for the preparation of a transdermal drug delivery composition. Generally, the process comprises (a) heating a mixture of a SMGA and a solvent to dissolve the gelling agent in the solvent, (b) cooling the mixture to form a gel, and (c) adding a drug which is suitable for transdermal delivery and a skin permeation enhancer to the mixture either before, during or after the heating, but prior to cooling. Optionally, a branching agent can be added to the mixture before, during or after the heating, but prior to cooling.

The temperature to which the mixture is heated should be higher than the dissolution temperature of the gelling agent in the solvent, and less than the boiling point of the solvent. For certain drugs, a lower temperature is beneficial as it reduces the possibility that the drug might degrade because of higher temperatures. The solvent used in the preparation of a small molecule gel preferably has a boiling point which is fairly high, for example from about 170° C. to about 200° C. In some examples, the dissolution temperature of the small molecule gelling agent in the solvent is from about 80° C. to about 100° C., and the mixture is heated to 120° C. to ensure the fast and complete dissolution of the small molecule gelling agent.

Upon cooling, the mixture comprising the SMGA and the solvent becomes supersaturated, at which point the small molecule gel is formed. The mixture is cooled or allowed to cool to form a gel (e.g. cooled to about 30° C., 20° C., 10° C., 0° C. or −10° C.). The cooling of the mixture can be effected, for example, by removal of the mixture from the heating source and by permitting the mixture to rest at ambient temperature, e.g. room temperature. The cooling step can also be carried out, for example, by placing the mixture in a water bath, an ice bath or a refrigerator. The optional presence of a branching agent during the cooling step can modify the crystallisation of the gelling agent, leading to the additional branching of the fibrils to give a more robust interconnecting network within the gel.

In an embodiment where the drug is dissolved in the solvent that forms the small molecule gel, the addition of the drug is preferably done prior or during the heating step. This insures that a maximum amount of the drug is present in the solvent, which increases the permeation rate of the drug through the skin.

Additives that do not significantly affect gelation or skin permeation can also be incorporated into the small molecule gel during preparation, to change certain characteristics of the small molecule gel, such as coloration.

The following examples are offered by way of illustration and not by way of limitation.

### EXAMPLES

### Example 1

### Preparation of a Haloperidol Transdermal Drug Composition comprising 5% GP-1 as SMGA

500 mg of GP-1, 30 mg of Haloperidol and 500 mg of linalool were added to propylene glycol to obtain 10 ml of solution. The ingredients were mixed, and the composition was heated for one hour at 120° C. to completely dissolve the Haloperidol. The mixture was then allowed to cool to ambient temperature. The obtained gel had a translucent appearance and it was white in color.

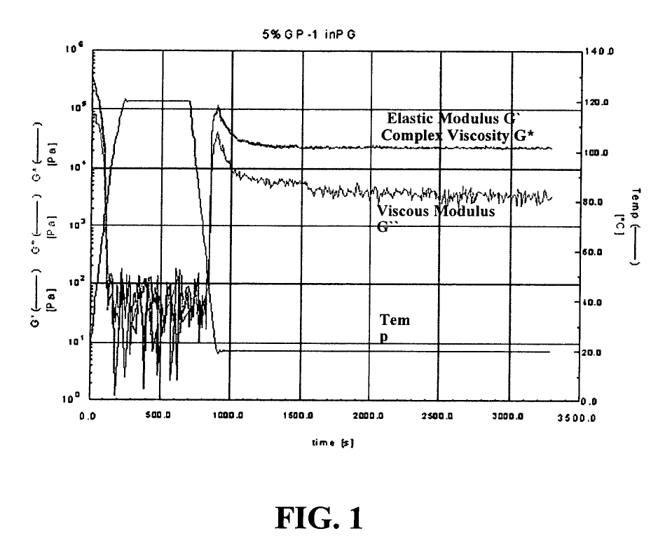

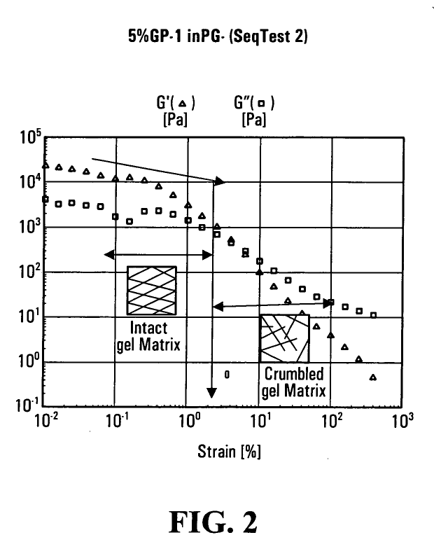

Viscosity tests were carried out on the obtained small molecule gel, the results of which are shown in FIGS. 1 and 2.

In FIG. 1, the results of a Dynamic Time Sweep experiment for the obtained small molecule gel are displayed. In a Dynamic Time Sweep experiment, the gel is heated to a specific temperature, for example 120° C., and it is then allowed to cool to a lower temperature, for example 20° C.

During the cooling period, the elastic and viscous moduli (G′ and G″) are recorded as a function of time. Futhermore, a value for G′ₘₐₓ can be obtained from the plateau portion of the curve of the response of the elastic and viscous moduli (G′ and G″) over time, which defines the quality of the gel. As showed in FIG. 1, the elastic and viscous moduli (G′ and G″) formed at 0.01% of strain, 20° C. and 1 Hz frequency are almost parallel to each other. This is characteristic of the mesh-like micro/nanostructure of a gel. These results confirm that a gel is indeed formed when a drug is dissolved in the solvent that forms the small molecule gel.

In FIG. 2, the stability of the prepared small molecule gel to strain is displayed. From FIG. 2, it can be seen that the small molecule gel can withstand up to 0.25% of strain. Below this strain level, the mesh-like micro/nanostructure remains intact, and above 0.25% of strain it crumbles. Knowing the strain threshold of the small molecule gel can be useful, for example to determine the package in which the gel will be stored and the storage conditions used to insure that the crumble strain is not applied to the gel prior to its use.

### Example 2

### Preparation of a Haloperidol Transdermal Drug Composition comprising 3% GP-1 as SMGA

The 3% GP-1 composition was prepared with a method similar to the one described in Example 1 above, but using 300 mg of GP-1 instead of 500 mg of GP-1 as SMGA.

### Example 3

### In Vitro Skin Permeation Experiments

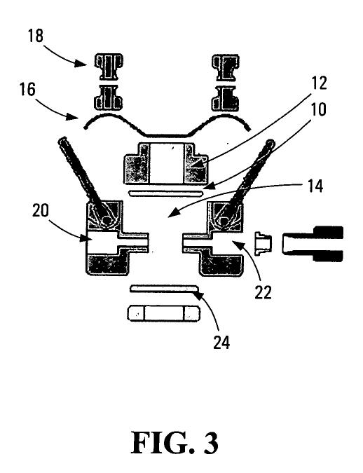

In vitro permeation studies of the transdermal drug delivery compositions were carried out using a flow-through cell method. A schematic of a flow-through cell is shown in FIG. 3. The flow-through cell comprises a donor compartment 12 and a receptor compartment 14, between which can be held a skin sample (e.g. human epidermis) 10. The donor compartment is secured with a clamping spring 16 and pressure setting screws 18. The receptor compartment solution can be collected through ports 20 and 22 and assayed by HPLC or other analytical methods, and the inside of the receptor compartment can be observed through viewing window 24.

Human epidermis from plastic surgery was mounted between the donor and receptor compartments of the flow-through cell, and about 1 ml of a small molecule gel was added to the donor compartment. A 0.03% lactic acid solution containing a 1% v/v antibacterial antimycotic solution was placed in a reservoir bottle, and the antibacterial and antimycotic solution was added to receptor solutions to maintain the integrity of the skin throughout the experiment and to minimize the microbial contamination in samples during the analysis. Cells were kept at 37±1° C. Cumulated receptor liquid samples were collected for every 6 hours and assayed by a reversed phase HPLC method.

In vitro skin permeation studies are used to verify the performance of the penetration of the drug through the stratum corneum. The following mathematical equation (I) can be used to relate the permeability coefficient (Kₚ), the lag time (Lt; Lt being equal to ⅙ D′), and the cumulative amount of drug permeated (Q).

$$Q = {AK}^{′} Co [ {D}^{′} t - \frac{1}{6} - \frac{2}{{π}^{2}} {\sum }_{n = 1}^{∞} \frac{{( - 1 )}^{n}}{{n}^{2}} {ⅇ}^{( - {D}^{′} {n}^{2} {π}^{2} t )} ] \qquad ( I )$$

Q is the cumulative amount of the drug permeated through the membrane with area ‘A’ in time ‘t’ from the donor solution at constant concentration ‘Co’ to the receptor phase at the sink condition. D′ is the diffusion parameter and K′ is the activity parameter. A non-linear regression analysis is used to estimate D′, K′ using the experimental results (Q and t). Permeability coefficient (Kₚ; Kₚ=D′K′) and the lag time are then calculated.

Solution of the above equation is further discussed in Okamato H, Komatsu H, Hashida M, Sezaki H., (1986), Effects of β-cyclodextrin and di-O-methyl-βcyclodextrin on the percutaneous absorption of butyl paraben, indomethacin and sulfanilic acid, Int. J. Pharm., 30:35-45, which is hereby incorporated by reference.

Using the results of the permeation experiments, the Kp and the lag time values for the compositions comprising 0, 3 and 5% (w/v) of GP-1 were obtained. These values are shown in Table 1.

TABLE 1
Kp and lag time (Lt) values for GP-1 compositions

| GP-1 | | | Kp*10³ | |
|---|---|---|---|---|
| Concentration | K′*10² | D′*10² | (cm/h) | Lt (h) |
| 0% | 6.06 ± 0.559 | 0.396 ± 0.0187 | 0.240 ± 0.0140 | 42.10 ± 1.96 |
| 3% | 9.76 ± 3.63 | 0.320 ± 0.0175 | 0.308 ± 0.0973 | 52.11 ± 2.89 |
| 5% | 16.12 ± 7.70 | 0.287 ± 0.528 | 0.436 ± 0.146 | 59.31 ± 10.83 |

The results from Table 1 show that low GP-1 concentrations (up to 5%) do not significantly affect the skin permeation rate of haloperidol. However, the lag time may be prolonged when the GP-1 concentration is increased. These conclusions are based on statistical tests, i.e., one-way ANOVA and pairwise comparison (Montgomery (2001) Design & Analysis of Experiments 5^{th} ed. Wiley). With an error rate of 0.05, the differences between permeation coefficients are not significant while the differences between lag times are significant.

### Example 4

### Preparation of a Haloperidol Transdermal Drug Composition comprising 1% D-DBS as SMGA

A 1% D-DBS composition was prepared with a method similar to the one described in Example 1 above, but using 100 mg of D-DBS instead of 500 mg of GP-1 as the SMGA, and 500 mg of farnesol instead of linalool as the skin penetration enhancers. Comparing the small molecule gels comprising 1% D-DBS as SMGA and the small molecule gels comprising 5% GP-1 as SMGA, it can be observed that the gels comprising D-DBS as SMGA (and at a lower concentration) provide a clearer gel.

### Example 5

### In Vitro Skin Permeation Experiments

A procedure similar to the one described in Example 3 above was carried out, except that the 1% D-DBS gel described in Example 4 was used instead of the GP-1 gels. From the results of the permeation experiments, compositions comprising 0.1% (w/v) of D-DBS were found to have a Kp value of 1.05*10⁻³ (cm/h) and a lag time value of 1.19 (hour).

All publications, patents and patent applications cited in this specification are herein incorporated by reference as if each individual publication, patent or patent application were specifically and individually indicated to be incorporated by reference. The citation of any publication is for its disclosure prior to the filing date and should not be construed as an admission that the present invention is not entitled to antedate such publication by virtue of prior invention.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A transdermal drug delivery composition comprising a small molecule gel, which small molecule gel comprises a small molecule gelling agent and a solvent, in which solvent is dissolved or suspended a drug that is suitable for transdermal delivery and a skin permeation enhancer.

2. The transdermal drug delivery composition according to claim 1, wherein the small molecule gelling agent has a molecular weight of 3000 g/mol or less.

3. The transdermal drug delivery composition according to claim 1, wherein the small molecule gelling agent has a molecular weight of 1000 g/mol or less.

4. The transdermal drug delivery composition according to claim 1, wherein the small molecule gelling agent has amide functional groups.

5. The transdermal drug delivery composition according to claim 1, wherein the small molecule gelling agent is N-laurylglutamic acid dibutylamide or 1,3:2,4-bis-O-benzylidene-D-sorbitol.

6. The transdermal drug delivery composition according to claim 1, wherein the small molecule gelling agent is present in a concentration of from about 0.1% w/v to about 20% w/v.

7. The transdermal drug delivery composition according to claim 1, wherein the small molecule gelling agent is present in a concentration of from about 4% w/v to about 15% w/v.

8. The transdermal drug delivery composition according to claim 1, wherein the drug is selected from haloperidol, norgestimate, and ethinyl estradiol.

9. The transdermal drug delivery composition according to claim 1, wherein the skin permeation enhancer is terpene or a terpenoid.

10. The transdermal drug delivery composition according to claim 1, wherein the skin permeation enhancer is selected from linalool, menthol, cineol and farnesol.

11. The transdermal drug delivery composition according to claim 1, wherein the small molecule gel comprises a branching additive.

12. The transdermal drug delivery composition according to claim 11, wherein the branching agent is selected from an ethylvinyl/ethylvinyl acetate copolymer or a poly-(methyl-vinyl ether)/maleic anhydride copolymer.

13. The transdermal drug delivery composition according to claim 11, wherein the branching agent is present in a concentration above 0.00.1% w/v.

14. The transdermal drug delivery composition according to claim 11, wherein the branching agent is present in a concentration of from about 0.001% w/v to about 0.1% w/v.

15. The transdermal drug delivery composition according to claim 1, wherein the solvent is propylene glycol.

16. The transdermal drug delivery composition according to claim 1, wherein the small molecule gel is biocompatible.

17. A process for the preparation of a transdermal drug delivery composition, the process comprising (a) heating a mixture of a small molecule gelling agent and a solvent to dissolve the small molecule gelling agent in the solvent; (b) cooling the mixture to form a gel; and (c) adding a drug that is suitable for transdermal delivery and a skin permeation enhancer to the mixture either before, during or after the heating, but prior to cooling.

18. The process according to claim 17, wherein a branching agent is added to the mixture either before, during or after the heating, but prior to cooling.
